(19) United States
(12) Patent Application Publication
(10) Pub. No.: US 2006/0099234 A1
(43) Pub. Date: May 11, 2006

(54) Implant containing cultured cartilage cells and method for producing this implant

(76) Inventor: Heinz Winkler, Krems (AT)

Correspondence Address: LERNER GREENBERG STEMER LLP, P O BOX 2480, HOLLYWOOD, FL, 33022-2480, US

(21) Appl. No.: 10/537,685

(22) PCT Filed: Dec. 5, 2003
(86) PCT No.: PCT/AT03/00362

§ 371 Date: Oct. 6, 2005

(30) Foreign Application Priority Data
Dec. 5, 2002 (AT) 1825/2002

(51) Int. Cl. A61K 35/12; A61F 13/00

(52) U.S. Cl. 424/422; 424/93.7

(57) ABSTRACT

In order to regenerate joint defects in the human or animal body, the invention provides an implant that is placed in the joint area. Said implant is comprised of a supporting body and, in fact, is particularly comprised of a spongy bone of human or animal origin, has infiltrating channels and, in one partial area, is impregnated (cartilage) cell suspension. This suspension penetrates into the cavities of the supporting body, the number of which preferably being increased by removing fats, cells, connective tissues, vessels or the like and into which cartilage cells settle whereupon forming a new cartilage tissue.

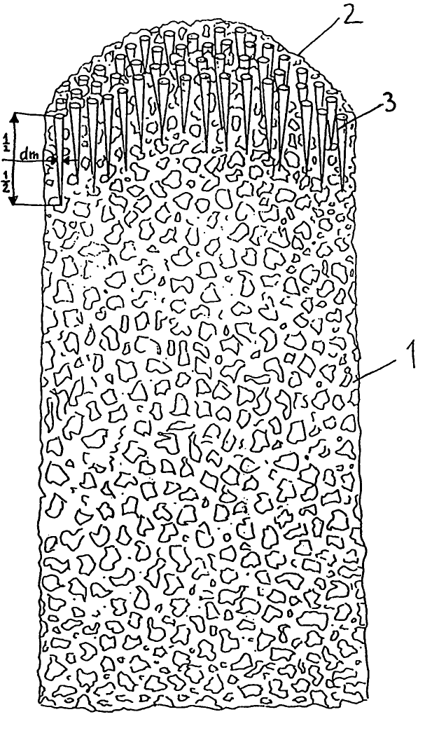

The invention concerns a new implant or such an implant body for regenerating joint defects in a human or animal body, which is formed of a supporting body (1) of a body tolerated material which comprises a pore, cell or spongy structure, particularly of a spongy bone of human or animal origin, and which is, at least partially, able to be impregnated or is impregnated with a suspension of tissue cells, a method for producing the new implant as well as its use for the above-mentioned purpose.

It is known for regenerating joint defects to culture cartilage tissue from autologic cells. These cells may be extracted from healthy cartilage sections by biopsy, but may also differentiated from mesenchymal stem cells, for example from bone marrow. Culture is made, in general, within a liquid nutrient. Implantation is usually performed by supplying a cell suspension which entrains considerable technical problems, but also the loss of cartilage cells and worsening of the result.

The present invention has the object to provide an implant which presents mechanical properties as similar to natural tissue as possible and guarantees a good connection to the ambient tissue, particularly to the subchondeal one, i.e. to the bone situated below the cartilage.

To solve this task, the invention suggests the use of a supporting body of a body tolerated material which comprises a pore or spongy structure, particularly of a spongy bone of human or animal origin, and which is, at least partially, impregnated with a cartilage cell suspension.

Thus, the subject matter of the invention is a novel implant or transplant or a new implant or transplant body of the type mentioned at the outset which is characterized in that it is provided with a supporting body having channels (3) beginning at a partial area of its surface (2) and ending within it for infiltrating the respective tissue cell suspension provided for regeneration.

According to claim 2, an impregnation of the supporting body with cartilage tissue is particularly preferred.

Such a supporting body has the advantage that it can easily be obtained and has a three-dimensional structure which provides ideal morphological conditions for the settlement of cells. By means of a supporting body prepared in such a way, the implant or transplant can be implanted precisely at the place of the defects to be treated in the region of a joint and can safely be fixed against changes of position or cell losses.

In order to make sure an optimum impregnation of the supporting body with the cell suspension, it is just that area, where a defect healing effect should be initiated, i.e. a corresponding partial area of the surface of the supporting body, where channels for the infiltration of the cell suspension should be provided and where they should begin.

At this point, the prior art in the field of implants and transplants should shortly be treated.

For example, from DE 40 40 872 A1 a tissue support to be implanted in the dental region is known onto which artificially bred, connective tissue-like cells having a desmodont character are applied for regeneration of the desmodont. As a tissue support, an enosseous implant, a flat, particularly membrane-like, support, a bone or a bone substituting material or a combination of such materials is considered there. The applied cells stem from the own tissue of the patient, of a donor tissue or of a specially cultured cell line which may be desmodontal cells or such cells, which differentiate after implantation of the tissue support to a tissue with a desmodont character.

About problems with introduction of the corresponding cell suspension, which in practice, as is known, occur again and again, is not reported there and, thus, not about possible approaches for solving such problems.

This applies substantially also for a method for preparing bones adapted for transplantation according to DE 961 654 A, where the impregnation of the bone tissue with cells is not to the fore, but rather the preparation of the bone material removing the soft tissue of it both internally and externally, of the fatty substances and of hemoglobin, the procedure being such that the bones are treated with protein removal agents, such as an at least 10% solution of H₂O₂ or a trypsin solution.

DE 41 21 043 relates to a bone substitute material which comprises one or more polypeptide(s) in a porous matrix that has (have) the biological effect of fibroblast growing factors. The healing behavior described therein corresponds to that of an autologic bone transplant.

Neither are the above-mentioned problems with introduction of the same kind of cell material into the bone substitute material nor are ways for solving them mentioned therein.

Finally, U.S. Pat. No. 4,553,272 relates to a repair material for human tissue on an implant basis where a porous or cellular inner structure of this material is suggested, the size of the pores or cells increasing starting from the surface. This US-A- too does not suggest any solution, which could come close to the present invention, for the problems with impregnating a cellular, porous or spongy material.

The infiltrating channels deepened into the new implants have preferably a diameter of about 300 to 500 μm, as may be seen from claim 3. They may be produced in a simple way either mechanically by boring or machining, by means of laser beams or by means of high-pressure water jets.

It is advantageous, if the supporting body is impregnated with the cellular suspension, preferably of cartilage cells, in a partial area only, i.e. where the cartilage should be formed or regenerated.

As may be derived from claim 3, the tapering cone-like shape of the infiltrating channels in the implant body are particularly preferred. By this shape, as has been found, it will be achieved that a substantially more intensive impregnation of the spongy bone material results in the region near the surface, when infiltrating the supporting body with the cartilage tissue cell suspension, than in the region of the tapering lower ends of the infiltrating channels. Due to the cone-like shape of these channels, a continuous transition of the intensity of impregnation with the suspension is ensured and, thus, it is achieved, that in the region of the surface substantially more cartilage tissue will be formed than in the region of the channel ends, so that a decreasing continuous gradient of the cartilage tissue proportion in the implant towards the interior will result, thus achieving a continuous transition from “predominantly cartilage tissue” to “predominantly bone tissue” which corresponds to the natural conditions or comes particularly close to them.

The tapered cone-like shape of the infiltrating channels has, moreover, the advantage that in the further particularly preferred embodiment of the invention described in the following, which consists in an aimed and, within the framework of the invention, particularly advantageous partial demineralization of the bone tissue, the bone material is more demineralized near the surface than in depth, which also results in the fact, that a more significant reduction of hard bone material will occur in the region of the surface than in the depth of the infiltrating channels so that the supporting body is more “bone-like” in the region of the surface than in the depth, the “cartilage-likeness” decreasing here also continuously towards the interior of the supporting body, while the bone-likeness increases correspondingly, which corresponds to the actual conditions in joints.

This applies also to the same extent for the infiltration of nutrient solutions and the like, as described later on.

The depth of the infiltration channels extends only over part of the thickness or thickness of material of the supporting body, and regarding this: vide claim 4. Particularly in cases where the infiltrating channels converge in the direction towards the interior of the supporting body, there is the advantage that a kind of sliding transition from cartilaginous to bony proportions of the implant is ensured.

According to another preferred embodiment of the invention following claim 5, the supporting body or implant body has a cylindrical shape, and the infiltration channels start preferably from its covering or basic surface or from one of its end surfaces.

According to claim 6 it is provided that the cover or basic surface of the implant cylinder, which comprises the infiltrating channels, is convex. In this way, anchoring thereof in corresponding cylindrical recesses of a bone is possible in a simple and optimum manner, and the convex front surface is, thus, in correspondence with the shape of the joint in an optimum fashion and permits, in this way, quick healing. Of course, it should be mentioned that the shape of the supporting body may also be adapted to any other shape or topography of the defect to be treated.

In order to improve the conditions for the cartilage growth, it is additionally advantageous and constitutes a particularly preferred variant of realization of the invention, if, in any case, that area of the implant body, which is to be impregnated or is impregnated with the (cartilage) cell suspension, is at least partially demineralized, as may be derived from claim 7, the advantage of a gradient-like, in depth decreasing demineralization being particularly given in the case of pointed cone infiltrating channels.

Concerning the method, which forms another important subject matter of the invention, for producing the implant whose basic conception and different variants of realization are described above, and being the preferred method for producing the implant, it is characterized according to claim 8 in that, starting from at least one partial area of its surface, infiltration channels are introduced or deepened into the supporting body (1), for example by mechanical boring, by a laser beam or by a pressurized water jet, after which at least this partial area of the supporting body (1) is introduced or immersed into a suspension which contains cultured cartilage cells.

The infiltrating channels favor penetrating of the suspension into the cellular porous or spongy structure of the supporting body and guarantee a complete impregnation in the respective partial area provided for it of the implant body.

The introduction of the cell suspension, as is preferred, may, in addition, be supported in that negative pressure or a vacuum is applied to the supporting body after immersing the infiltrating channels of the supporting body into the cell suspension, as is also disclosed in claim 8.

In an advantageous manner, see claim 9 in this context, at least the area to be impregnated is subjected to a cleaning and/or demineralization procedure before impregnating. By the cleaning procedure, marrow matter, such as fats, different other cells, connective tissue, vessels or the like are removed, thus increasing the number of pores and cavities wherein the cells introduced by the cell suspension, particularly cartilage cells, may settle, e.g. by adding appropriate nutrients.

Furthermore, there is the advantage that the mechanical properties of the support become more “cartilage-like” by demineralization, thus improving the conditions for the cartilage cell growth.

In order to effect demineralization, at least the area to be impregnated of the supporting body, according to an advantageous variant of claim 9, is introduced or immersed into a demineralization medium, for example hydrochloric acid, particularly of a concentration of around 0.5 mol/L, after which it is preferred to apply negative pressure or a vacuum to the supporting body, thus ensuring a particularly complete impregnation thereof with the cell suspension. Afterwards, the demineralization medium, and the salts contained therein and being eliminated from the bone, are removed by rinsing.

In order to favor adherence in the supporting body and the growth of the respective cells, particularly of cartilage cells, the area to be impregnated of the supporting body may be impregnated with a nutrient, for example hyaluronic acid or collagen, as is disclosed in claim 10. This impregnation may be performed before soaking the supporting body with the (cartilage) cell suspension.

Alternatively, it may be provided to perform simultaneously an impregnation of the supporting body with a mixture of a cell or cartilage cell suspension and with a medium that favors the development and breeding of cells, such as hyaluronic acid or collagen.

The use of an implant or implant or implant body according to the invention or produced in accordance with the invention according to claim 11 forms a further important subject matter of the invention.

In this connection, an arrangement of the infiltrating channels according to claim 12 is preferred.

In the drawing which clarifies the invention, an implant according to the invention is schematically illustrated. This is based on a supporting body 1 which, in the case illustrated, consists of a spongy bone of either human or animal origin. The supporting body has here a cylindrical shape, the end or cover surface 2 of this cylindrical supporting body 1 being vaulted to a convex shape. From this convex end surface 2, infiltrating channels 3, which are oriented parallel to each other and spaced from each other by more than the dimension of their diameter, extend in the direction towards the interior of the supporting body 1, and taper, beginning from the convex cover surface 2 of the supporting body 1 in the direction towards the interior of the supporting body, in a pointed cone like manner. The center diameter dm of the channels 3 may amount to between about 300 and 500 μm, the distance of the channels 3 from each other amounts, for example, to about 1 to 3 mm. The depth of the channels 3 is chosen in such a manner that a respective desired cartilage thickness is obtained at the implantation place.

Manufacture of the channels 3 can be made by means of a mechanical drill bit, but also by means of boring with a laser beam or with a water jet.

Having produced the infiltrating channels, the supporting body, which consists of a spongy bone material, is cleaned and demineralized, particularly by hydrochloric acid, 0.5 mol/l.

Cleaning is done by a series of bathes, preferably in degreasing solutions so that fats, different cells, connective tissue and vessels are removed from the bone material so that additional cavities for the settlement of the (cartilage) cells to be infiltrated are created.

For this demineralization, the supporting body 1 together with its end region comprising the channels 3 are introduced into a bath of hydrochloric acid, and a vacuum is applied to the supporting body 1, thus supporting permeation of the solution of hydrochloric acid into the cellular, porous or spongy structure of the supporting body. Both the vacuum and the duration of demineralization are chosen in such a way that an optimum cartilage-like consistency of the bone is achieved.

Subsequently, the thus pretreated supporting body 1 may be impregnated with a nutrient, which favors the development of cartilage cells, for example with hyaluronic acid or with collagen.

After this pretreatment, the supporting body is impregnated with the (cartilage) cell suspension. To this end, that area of the supporting body is immersed into a cartilage cell suspension, usual per se, containing cartilage cells removed beforehand by biopsy, where the cartilage tissue shall develop. If appropriate nutrients are used, even mesenchymal stem cells, for example from the bone marrow or from umbilical chord blood, could be used.

By applying negative pressure or vacuum, permeation of the cell suspension along the infiltrating channels into the trabecular spaces is ensured. Alternatively, the cell suspension may also be combined with the nutrient respectively chosen, and the mixture may be introduced into the supporting body by the aid of vacuum.

Subsequently, one waits for the suspension being sucked into the spongy structure of the supporting body and adhering to the surfaces of the pores. By the nutrient, further spreading of the cells afterwards is ensured and the development and multiplying of the basic substance of cartilage is stimulated. As soon as the desired portion of the supporting body is filled with cartilage tissue, the preparation is terminated.

In the course of the settlement, the trabecular structure may be dissolved step-by-step by adding collagenagen or other lytic enzymes so that, finally, space is created for a complete substitution of the supporting body by a cartilage matrix developed by the cells.

The implant, thus obtained, can then be implanted in a defect joint region, wherein first cylindrical recesses are worked into the bone and are provided for insertion of the implant, into which a corresponding implant body is snug fittingly inserted. By appropriately dimensioning the cylinder of the supporting body in the bone (a diameter about 0.5 to 1 mm smaller), a snug fit is obtained with the bony portion of the implant. The cartilaginous portion of the implant is finally brought precisely to the joint level.

In summary, the advantage of the present invention shall be emphasized again:

An important aspect of the invention is that by the new method, it is the first time that an autologic cartilage can be produced for transplantation purposes which corresponds almost completely to a natural one. In this connection, it is essential that the transplant has as much the same shape as possible as the natural joint coating already when incorporating it. In nature, the cartilage is firmly connected to or indented with the bone below. A similar indentation is achieved by the new method. Thus, a transplant will be obtained which comprises a natural ratio between the proportions of bone and cartilage already when incorporating it. The new implant or transplant is cartilaginously soft at the joint surface and turns towards the depth continuously into a bony hard material, just as it is the case in natural surroundings. This continuous transition can be obtained by a different intensity of demineralization, and this, in turn, by a differently strong attack of the hydrochloric acid. This, as well as the differently intensive permeation of the cell suspension is achieved by tapering the bored channels, as described.

1-12. (canceled)

13. An implant for regenerating joint defects in a human or animal body, comprising:
a supporting body of a body-tolerable material having a porous or spongy structure;
said supporting body having infiltration channels formed therein beginning from a surface thereof and ending in said body, said infiltration channels receiving therein a tissue cell suspension for regeneration.

14. The implant according to claim 13, wherein said supporting body comprises a spongy bone of human or animal origin.

15. The implant according to claim 13, wherein said supporting body is impregnated with a cartilage tissue cell suspension.

16. The implant according to claim 13, wherein said infiltration channels have a hollow cylindrical shape and a diameter in a range from 300 to 500 μm.

17. The implant according to claims 16, wherein said infilration channels have a depth substantially 3 to 10 times said diameter.

18. The implant according to claim 17, wherein a value of said depth is approximately 5-fold to 10-fold a value of said diameter.

19. The implant according to claim 13, wherein said infiltration channels start from said surface of said supporting body and taper inwardly in a pointed cone shape or pointed frustum shape, and each of said infiltration channels have a midway diameter in a range from 200 to 500 μm.

20. The implant according to claims 19, wherein said infiltration channels have a depth substantially 3 to 10 times said midway diameter.

21. The implant according to claim 20, wherein a value of said depth is approximately 5-fold to 10-fold a value of said diameter.

22. The implant according to claim 13, wherein said supporting body has a cylindrical shape with a base and a cover surface, and said infiltration channels start out from at least one of said base and said cover surface.

23. The implant according to claim 22, wherein said at least one of said base and said cover surface at which said infiltration channels begin is vaulted in a convex shape.

24. The implant according to claim 14, wherein the part of said supporting body impregnated with cartilage cell suspension is at least partially demineralized.

25. A method of producing an implant or implant body for regenerating joint defects in a human or animal body, the method which comprises:
providing a supporting body and, starting from at least one partial area of a surface thereof, forming infiltration channels into the supporting body;
subsequently introducing or immersing at least the one partial area of the supporting body into a suspension containing cultured cells for regenerating cell tissue.

26. The method according to claim 25, which comprises impregnating the supporting body with cultured cartilage cells for regenerating cartilage tissue.

27. The method according to claim 25, which further comprises, subsequently to introducing the supporting body into a cell suspension, subjecting the supporting body to a vacuum.

28. The method according to claim 25, wherein the forming step comprises forming voids in the supporting body by one of mechanical boring, laser beam cutting, a water jet boring.

29. The method according to claim 25, which further comprises, prior to impregnation with a cell or cartilage cell suspension, subjecting an area of the supporting body to be impregnated to a cleaning and/or demineralizing procedure.

30. The method according to claim 29, which comprises introducing at least the area to be impregnated into an acidiferous demineralization medium, subjecting to a vacuum relative to ambient pressure, and subsequently rinsing the body for removing therefrom demineralization medium and salts, contained therein and being eliminated from the bone by the demineralization.

31. The method according to claim 30, which comprises demineralizing with 0.5 N hydrochloric acid.

32. The method according to claim 25, which comprises, prior to impregnating with the cell or cartilage cell suspension, soaking at least the area of the supporting body to be impregnated with a nutrient favoring a development of cells or cartilage cells.

33. The method according to claim 32, which comprises soaking with hyaluronic acid or collagen.

34. The method according to claim 25, which comprises impregnating the supporting body with a mixture of a cell or cartilage cell suspension and a medium favoring a development of cells or cartilage cells.

35. The method according to claim 25, which comprises impregnating the supporting body with a mixture of a cell or cartilage cell suspension and hyaluronic acid or collagen for favoring a development of cells or cartilage cells.

36. A method of regenerating joint defects in a human or animal body, which comprises:
providing an implant according to claim 13 containing cultured cells for regenerating joint defects in a human or animal body;
forming at least one recess in a region of a bone forming the joint to be regenerated; and
inserting the implant into the recess in the bone for regenerating the joint defect.

37. The method according to claim 36, which comprises providing an implant impregnated with cultured cartilage tissue cells.

38. The method according to claim 36, wherein the forming step comprises forming a plurality of recesses, arranged in mosaic form, in the implant region of the bone, and inserting a respective said implant according to claim 13 into each of the recesses.
